(19) United States
(12) Patent Application Publication
(10) Pub. No.: US 2025/0144216 A1
(43) Pub. Date: May 8, 2025

(54) NEAR-INFRARED PHOTODYNAMIC THERAPY USING SECOND HARMONIC GENERATION NANOPARTICLE AND VISIBLE LIGHT REACTIVE PHOTOSENSITIZER

(71) Applicant: ARIEL SCIENTIFIC INNOVATIONS LTD., Ariel (IL)

(72) Inventors: Refael MINNES, Ariel (IL); Ayan BARBORA, Ariel (IL)

(21) Appl. No.: 18/838,137

(22) PCT Filed: Feb. 14, 2023
(86) PCT No.: PCT/IL2023/050156

§ 371 Date: Aug. 13, 2024

Related U.S. Application Data
(60) Provisional application No. 63/309,652, filed on Feb. 14, 2022.

(51) Int. Cl. A61K 41/00; A61K 47/60; A61K 47/69

(52) U.S. Cl. CPC A61K 41/0057; A61K 47/60; A61K 47/6929

(57) ABSTRACT

The present invention is directed to second harmonic generation particle comprising a photosensitizer spaced by a non-ionic water-soluble spacer, method of manufacturing thereof and use thereof such as for photodynamic therapy.

### CROSS REFERENCE TO RELATED APPLICATIONS

This application claims the benefit of priority to U.S. Provisional Patent Application No. 63/309,652 filed Feb. 14, 2022, entitled “NEAR-INFRARED PHOTODYNAMIC THERAPY USING SECOND HARMONIC GENERATION NA-1 NOPARTICLE AND VISIBLE LIGHT REACTIVE PHOTOSENSI”, the contents of which are incorporated herein by reference in their entirety.

### FIELD OF THE INVENTION

The present invention is in the field of nanoparticles including a photosensitizer in operable communication with a second harmonic particle, such as for use in photodynamic therapy.

### BACKGROUND

Photodynamic therapy (PDT) uses light and a chemical substance called photosensitizer (PS) to generate molecular oxygen and stimulate cell death. This phenomenon is called phototoxicity. This promising approach has been development for various applications including microbial inactivation and treatment of infections. PDT as a treatment procedure is advantageous over conventional therapies as it provides dual specificity: 1) PS are targeted to specific location of cells or tissues and 2) irradiation regimes can be spatially confined to allow selective treatment. The production of reactive oxygen species (ROS) in individual cells results in irreversible damage and cell death. PDT directly initiates apoptotic response in targeted cell without requiring intermediate signal transduction pathways, as is the case in drug-resistant neoplastic cells.

Second harmonic generation (SHG) is a nonlinear optical effect, in which two photons are absorbed and converted to emit one photon with double the energy (and half the wavelength) when passing through certain materials. Being a non-parametric process, SHG effect can be tuned to achieve localized excitation. With the absence of energy loss involved in SHG, photobleaching is practically avoided. This suggests SHG can be effectively used to generate specific biological responses in targeted applications without affecting surrounding healthy tissue.

Absorption spectra of conventionally used PS molecules range within 400-750 nm (visible spectrum). However, light has its maximum depth of penetration into a tissue is within the range of 650-950 nm termed as the near-infrared (NIR) window. Accordingly, there is a need to develop means for inducing PDT within the target region of subject's organism by utilizing the NIR window excitation wavelength.

### SUMMARY

The following embodiments and aspects thereof are described and illustrated in conjunction with systems, tools and methods which are meant to be exemplary and illustrative, not limiting in scope.

In one aspect, a composition comprising a plurality of nanoparticles, wherein each of the plurality of nanoparticles comprises (i) a second harmonic generation (SHG) particle, (ii) a non-ionic water-soluble spacer, and (iii) a photosensitizer; wherein: the SHG particle is configured to emit a plurality of photons at a wavelength range of between 360 and 450 nm; the photosensitizer is a water insoluble photosensitizer characterized by an absorbance range at least partially overlapping with the wavelength range; and wherein the photosensitizer is in operable communication with the SHG particle to allow excitation of the photosensitizer by the plurality of photons.

In some embodiments, the operable communication comprises a distance between the SHG particle and the photosensitizer suitable for excitation of the photosensitizer by the plurality of photons.

In some embodiments, the SHG particle and the photosensitizer are spaced by the non-ionic water-soluble spacer; and wherein the distance is predetermined by the molecular weight of the water-soluble spacer.

In some embodiments, the molecular weight is between 50 and 1000 Da.

In some embodiments, the water-soluble spacer comprises ethylene glycol (EG), EG oligomer, or polyethyleneglycol (PEG).

In some embodiments, the SHG nanoparticle is selected from, barium titanate, lithium niobate and potassium niobate, or any combination thereof.

In some embodiments, the photosensitizer is selected from curcuminoid-based photosensitizer, porphyrin-based photosensitizer or phthalocyanines-based photosensitizer, and any combination thereof.

In some embodiments, the porphyrin-based photosensitizer is protoporphyrin IX including any salt thereof.

In another aspect, there is provided a composition comprising a plurality of nanoparticles, wherein each of the plurality of nanoparticles has a core in contact with a shell; wherein: the core comprise a SHG particle configured to emit a plurality of photons at a wavelength range of between 360 and 450 nm; the shell comprises a water-soluble non-ionic spacer and a photosensitizer; and the photosensitizer is a water insoluble photosensitizer characterized by an absorbance range at least partially overlapping with the wavelength range; the photosensitizer is spaced from the core by water-soluble non-ionic spacer to allow excitation of the photosensitizer by the plurality of photons.

In some embodiments, the photosensitizer is selected from curcuminoid-based photosensitizer or porphyrin-based photosensitizer, and any combination thereof.

In some embodiments, the porphyrin-based photosensitizer is protoporphyrin IX including any salt thereof.

In some embodiments, the water-soluble non-ionic spacer has an average molecular weight between 50 and 1000 Da.

In some embodiments, the water-soluble non-ionic spacer comprises ethylene glycol (EG), EG oligomer, or polyethyleneglycol (PEG).

In some embodiments, a weight ratio between the photosensitizer and the non-ionic water-soluble spacer within the composition is between 1:150 and 1:3500.

In some embodiments, a weight ratio between the photosensitizer and the SHG particle within the composition is between 5:1 and 1:5.

In some embodiments, the plurality of nanoparticles is suitable for inducing a photodynamic response in a cell.

In another aspect, there is provided a composition comprising a plurality of nanoparticles, wherein each of the plurality of nanoparticles comprises: (i) a SHG particle, (ii) PEG and (iii) a photosensitizer; wherein: the SHG particle and the photosensitizer are spaced by the PEG; the photosensitizer is a water insoluble photosensitizer characterized by an absorbance at a wavelength in a range between about 360 and 450 nm; the SHG particle is barium titanate; and the PEG is characterized by an average molecular weight between 50 and 1000 Da.

In some embodiments, the photosensitizer is selected from protoporphyrin IX and curcumin, including any salt thereof; and wherein the PEG is characterized by an average molecular weight of between about 280 and 320 Da.

In another aspect, there is provided a pharmaceutical composition comprising the composition of the invention and a pharmaceutically acceptable carrier.

In another aspect, there is provided a method for killing a cell of interest in a subject, comprising (i) administering an effective amount of the pharmaceutical composition of the invention to the subject, thereby internalizing the nanoparticle into the cell of interest; (ii) irradiating the cell of interest at a wavelength suitable for excitation of the barium titanate, thereby inducing death of the cell of interest.

In some embodiments, step (ii) comprises irradiating a tissue of the subject comprising the cell of interest.

In some embodiments, the subject is afflicted with a pathogen-related disease, a cell proliferation related disease, or any combination thereof.

In some embodiments, the cell of interest comprises a cancer cell, a pathogenic cell, a cell infected by a pathogen, an immune cell or an activator thereof, or any combination thereof.

In another aspect, there is provided a method for treating a disease or disorder within a subject, comprising (i) administering to the subject a therapeutically effective amount of the pharmaceutical composition of the invention; and (ii) irradiating a tissue of interest of the subject at a wavelength suitable for excitation of the barium titanate, thereby treating the disease or the disorder.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a scheme presenting the photodynamic effect responsive to NIR light involving harmonic nanoparticle conjugated to photosensitizer (HNP-PS) using polyethylene glycol (PEG).

FIGS. 2A-2B are graphs presenting 2A. Absorbance and 2B. Fluorescence spectra of BT (Barium Titanate), Protoporphyrin IX (PPIX), Curcumin (Crcmn), BT-PPIX (Barium Titanate conjugated with PPIX) and BT-Crcmn (Barium Titanate conjugated with Curcumin).

FIGS. 3A-3C are graphs presenting emission spectra of 3A. BT (Barium Titanate), 3B. Protoporphyrin IX (PPIX), and 3C. Curcumin (Crcmn); box highlights the Second Harmonic Generation 3B. Protoporphyrin IX (PPIX) vs. BT+PPIX (Barium Titanateconjugated with PPIX) and 3C. Curcumin (Crcmn) vs. BT+Crcmn (Barium Titanate conjugated with Curcumin) after excitation with Near Infrared (800 nm) femtosecond pulsed wave laser beamline.

FIGS. 4A-4B are graphs bars presenting 4A. Viability of S. aureus after dark treatment with BT (Barium Titanate) or PPIX (Protoporphyrin IX) or Crcmn (Curcumin) or HNP-PS conjugates. 4B. Viability of S. aureus after photodynamic treatment using pulsed NIR laser with either BT+PPIX or BT+Crcmn or HNP and PSs alone normalized to constant power across all irradiated samples with reference to that of laser alone. Cell counts across all labels were kept constant. The error bars indicate standard errors of 6 samples each of HNP-PS conjugates with laser irradiation, 4 samples each of HNP-PS conjugates in dark and 2 samples of each of the rest. Each label represents data for constant cell (200 μl of 10³-10⁴ cells/ml) and nanoparticle/photosensitizer (20 μl of 400 μg/ml) concentrations as detailed in methods.

FIG. 5A-5B. Spectral Emission upon Pulsed 800 nm laser excitation of 5A. BT+Crcmn and 5B. BT+PPIX. Samples were used at the same concentration of indicated perovskite or photosensitizer or both and irradiated under the same laser power conditions.

FIG. 6. Percent viability of MCF7 cells after PDT (photodynamic therapy) using either BT+Cremn or BT+PPIX as drug conjugated using PEG 100 and pulsed NIR laser excitation. Values represent mean and standard deviation from 8 measurements.

### DETAILED DESCRIPTION

According to some embodiments, there is provided a composition comprising a plurality of particles, wherein each of the plurality of particles (also referred to herein as the “nanoparticle of the invention”) comprises: (i) a second generation (SHG) particle, (ii) a non-ionic water-soluble spacer and (iii) a photosensitizer; and wherein the SHG nanoparticle is configured to emit a plurality of photons at a wavelength range of between 360 and 450 nm; and the photosensitizer is a water insoluble photosensitizer characterized by an absorbance range at least partially overlapping with the wavelength range; and wherein the photosensitizer is in operable communication with the SHG particle to allow excitation of the photosensitizer by the plurality of photons.

As used herein, the term “second harmonic generation particle” refers to nanoparticles capable of absorbing photons and converting two absorbed photons into a single emitted photon (having double the energy). SHG is capable of inducing excitation of a photosensitizer in operable communication therewith.

In some embodiments, the nanoparticles of the invention has a core in contact with a shell; wherein: the core comprise a SHG particle configured to emit a plurality of photons at a wavelength range of between 360 and 450 nm; and wherein, the shell comprises a water-soluble non-ionic spacer and a photosensitizer; and the photosensitizer is a water insoluble photosensitizer characterized by an absorbance range at least partially overlapping with the wavelength range; the photosensitizer is spaced from the core by water-soluble non-ionic spacer to allow excitation of the photosensitizer by the plurality of photons.

In some embodiments, the nanoparticle of the invention has a core in contact with a shell; wherein: the core comprise a SHG particle configured to emit a plurality of photons at a wavelength range of between 360 and 450 nm; and wherein, the shell comprises a water-soluble non-ionic spacer and a photosensitizer; and the photosensitizer is characterized by an absorbance range at least partially overlapping with the wavelength range; the photosensitizer is spaced from the core by a water-soluble non-ionic spacer to allow excitation of the photosensitizer by the plurality of photons.

In some embodiments, the nanoparticle of the invention comprises a single SHG particle. In some embodiments, the nanoparticle of the invention comprises a plurality of SHG particles. In some embodiments, the SHG particle is a nanoparticle. In some embodiments, the SHG particle is characterized by an average particle size in a range between 10 to 200 nm, between 10 and 30 nm, between 10 and 50 nm, between 30 and 70 nm, between 50 and 100 nm, between 100 and 150 nm, between 150 and 200 nm, including any range in between.

In some embodiments, the SHG particle is or comprises a perovskite. In some embodiments, the plurality of SHG nanoparticle is barium titanate, lithium niobate, potassium niobate or any combination thereof. In some embodiments, the SHG particle is in a crystalline state. In some embodiments its crystallinity degree is between 70 and 99.9%, between 70 and 80%, between 80 and 90%, between 90 and 99.9%, between 95 and 99.9%, including any range in between. As used herein the term “perovskite” refers to any material having a crystal lattice of a corner sharing BX6 octahedra crystalized with a chemical formula ABX3. A representing the cation, B the anion and X usually an oxide, can also be halides.

In some embodiments, the SHG particle is characterized by absorbance in a range between 600 to 900 nm, between 600 and 650 nm, between 650 and 700 nm, between 700 and 750 nm, between 750 and 800 nm, between 800 and 850, between 850 and 900 nm, between 900 and 950 nm, between 950 and 1000, between 1000 and 1200 nm. In some embodiments, the SHG particle is characterized by absorbance in a range between 750 and 850 nm, between 750 and 770 nm, between 770 and 790 nm, between 790 and 810 nm, between 810 and 830 nm, between 830 and 850 nm, including any range in between. In some embodiments, the SHG particle has at least one absorbance peak in the range between 600 to 900 nm.

It is postulated that the absorbance wavelength of the SHG particle is at a wavelength range with maximum light penetration depth in the biological tissue. Thus, it should be apparent that the particle of the invention is usually located within the subject's organism. Accordingly, in order to efficiently induce photo dynamic therapy (PDT) inside the subject's organism (as opposed to PDT generation within the skin of the subject), the particle of the invention (in particular the SHG particle) needs to be capable of absorbing light in a wavelength range between 600 to 900 nm, which is the wavelength range with the greatest penetration depth in the biological tissue.

As used herein, the term “plurality” encompasses any integer equal to or greater than 2. In some embodiments, a plurality comprises at least 2, at least 3, at least 4, at least 5, at least 6, at least 7, at least 8, at least 9, or at least 10, or any value and range therebetween. Each possibility represents a separate embodiment of the invention.

In some embodiments, the term “operable communication” encompasses a distance between the SHG particle and the photosensitizer sufficient for inducing excitation of the photosensitizer by the plurality of photons emitted by the SHG particle. The term “distance” refers to the average distance between the SHG particle and the photosensitizer. In some embodiments, the distance between the SHG particle and the photosensitizer within the nanoparticle of the invention is between 8 A (Angstrom) and 100 nm, between 8 A and 20 nm, 8 A and 10 nm, between 1 and 5 nm, between 1 and 8 nm, between 8 nm and 10 nm, between 10 and 30 nm, between 30 and 50 nm, between 50 and 100 nm, and at most 100 nm, at most 80 nm, at most 50 nm, at most 30 nm, at most 10 nm, at most 8 nm, at most 1 nm, including any range in between. In some embodiments, the photosensitizer is in operable communication with the SHG particle, so as to induce generation of reactive oxygen species (ROS).

In some embodiments, the term “operable communication” encompasses a distance suitable for inducing ROS generation (by the photosensitizer) upon irradiation of the nanoparticle of the invention at a wavelength between 600 and 900 nm. In some embodiments, operable communication refers to a distance sufficient for inducing generation of ROS sufficient for use thereof in photodynamic therapy. In some embodiments, the nanoparticle of the invention is suitable for inducing a photodynamic response in cell. In some embodiments, the term “operable communication” encompasses a quantum efficiency of the nanoparticle of the inveniton sufficient for inducing generation of ROS. The term “quantum efficiency” refers to the ration between the photons emitted during the irradiation of the nanoparticle of the inveniton and the photons absorbed by the photosensitizer. In some embodiments, the quantum efficiency of the nanoparticle of the inveniton is between 10 and 90%, between 10 and 50%, between 10and 80%, between 50 and 90%, between 30 and 90%, between 30 and 50%, between 10and 30% including any range in between.

It is postulated that when the SHG particle and the photosensitizer are in direct contact with each other, the SHG particle undergoes non-radiative decay upon excitation thereof, and thus the SHG particle can't induce excitation of the photosensitizer. Furthermore, it is postulated that when the distance between the SHG particle and the photosensitizer is above the distance disclosed hereinabove, the photons emitted the SHG particle don't reach the photosensitizer, so that the photosensitizer is not excited by the SHG particle.

In some embodiments, the SHG particle and the photosensitizer are spaced or distanced by the non-ionic water-soluble spacer (also referred to as herein as “spacer”) within the nanoparticle of the inveniton, so as to provide the SHG particle and the photosensitizer in operable communication with each other. In some embodiments, the SHG particle and the photosensitizer are spaced by the non-ionic water-soluble spacer, so that the distance between the SHG particle and the photosensitizer within the nanoparticle of the invention is as described hereinabove (e.g., between 8 A and 100 nm). In some embodiments, the space is predetermined by the spacer molecular weight.

In some embodiments, the photosensitizer is a water insoluble photosensitizer. In some embodiments the photosensitizer is selected from protoporphyrin-based, curcumin-based, or phthalocyanine-based photosensitizer including any combination thereof. In some embodiments, the photosensitizer is characterized by an absorbance at a wavelength in a range between 360 and 450 nm, between 360 and 390 nm, between 360 and 400 nm, between 370 and 380 nm, between 370 and 450 m, between 410 and 430 nm, between 400 and 450 nm and any combination thereof.

In some embodiments, the spacer is a small molecule (below 500 Da, below 300 Da, below 100 Da), a polymer or an oligomer, including any combination thereof. In some embodiments, the spacer is characterized by an average length between 8 A and 100 nm, between 8 A and 10 nm, between 1 nm and 10 nm, between 1 and 20 nm, between 10 and 20 nm, between 5 and 20 nm, between 20 and 40 nm, between 40 and 60 nm, between 60 and 80 nm, between 80 and 100 nm, including any range in between, wherein the average length refers to the actual length of the assembled spacer in the nanoparticle of the invention.

In some embodiments, the spacer has a weight average molecular weight below 1000, below 800, below 600, below 400, below 200 Da, below 150 Da, and between 50and 1000 Da, between 50 and 150, between 75 and 125 Da, between 50 and 200 Da, between 200 and 400, between 250 and 350 Da, between 400 and 600 Da, between 600and 800 Da, between 800 and 1000 Da, including any range in between. As used herein, the term “weight average molecular weight” generally refers to a molecular weight measurement that depends on the contributions of polymer molecules according to their sizes.

In some embodiments, the spacer is characterized by a water solubility of at least 50 mg/ml, at least 10 mg/ml, at least 20 mg/ml, at least 30 mg/ml, and between 10 and 1000 mg/ml, between 10 and 30 mg/ml, between 30 and 70 mg/ml between 50 and 100 mg/ml, between 100 and 200 mg/ml, between 200 and 400 mg/ml, between 400 and 600 mg/ml, between 600and 800 mg/ml, between 800 and 1000 mg/ml, including any range in between, at a defined temperature range (e.g., room temperature, at 20 to 100° C).

Non limiting examples of the non-ionic water-soluble spacer include but are not limited to: a glycol (such as ethylene glycol (EG), propylene glycol, etc.); a glycol (e.g. EG) oligomer (i.e. comprising between 2 and 5, 2, 3, 4, or 5 glycol (e.g. EG) monomeric units), polyalkylenoxide (e.g. PEG, PPG, etc.), polyethylene glycol (PEG), polyvinyl alcohol, polyacrylamide, poly (propylene oxide), including any combination thereof.

In some embodiments, the non-ionic water-soluble spacer is selected from EG, EG oligomer, and PEG, and is characterized by an average MW between about 50 and about 300 Da, between about 50 and about 1000 Da, between about 50 and about 500 Da, between about 50 and about 200 Da, between about 50 and about 150 Da, between about 50 and about 400 Da, between about 50 and about 600 Da, including any range between.

In some embodiments, the nanoparticle of the invention consists essentially of the SHG particle, the spacer, and the photosensitizer. In some embodiment, a w/w percentage of the SHG particle, the spacer and the photosensitizer within the nanoparticle of the invention is at least 85%, at least 90%, at least 95%, at least 99%, and between 85and 99%, between 85 and 90%, between 87 and 93%, between 90 and 95%, between 95and 99%, including any range in between.

In some embodiments, the spacer and the photosensitizer are bound to the SHG particle. In some embodiments, the bond is a non-covalent bond and/or physical interactions. In some embodiments, non-covalent bond is hydrogen bond, van-der-Waals bond, dipole-dipole interactions, hydrogen bond, London forces or any combination thereof.

In some embodiments, a weight ratio between the photosensitizer and the spacer within the nanoparticle of the invention is between about 1:100 and 1:4000, about 1:150 and about 1:3500, about 1:166 and 1:3333, between about 1:166 and 1:3000, between about 1:166 and 1:2000, between about 1:166 and 1:1000, between about 1:166 and 1:500, including any range in between.

In some embodiments, a molar ratio between the spacer and the SHG particle within the nanoparticle of the invention is between 1:0.005 and 1:0.05, between 1:0.002 and 1:0.1, between 1:0.005 and 1:0.01, between 1:0.01 and 1:0.05, between 1:0.01 and 1:0.1, between 1:0.007 and 1:0.0085, between 1:0.005 and 1:0.01, between 1:0.007 and 1:0.01, between 1:0.07 and 1:0.09, between 1:0.01 and 1:0.05, including any range in between.

In some embodiments, a w/w percentage of the SHG particle within the nanoparticle of the invention is between 0.5 and 50%, between 0.5 and 10%, between 0.5 and 20%, between 15 and 25%, between 10 and 50%, between 10 and 30%, between 10 and 20%, between 20 and 30%, between 30 and 40%, between 40 and 50%, including any range in between.

In some embodiments, a w/w percentage of the SHG particle within nano particle of the invention is between 0.5 and 15%, between 0.5 and 2%, between 0.5 and 5%, between 0.5 and 10%, between 5 and 15%, between 10 and 15%, including any range in between.

In some embodiments, a w/w percentage of the spacer within the nanoparticle of the invention is between 40 and 99%, between 50 and 70%, between 60 and 99%, between 80 and 99%, between 90 and 99%, between 90 and 95%, between 95 and99%, including any range in between.

In some embodiments, a weight ratio between the photosensitizer and the SHG particle is between 5:1 and 1:5, between 5:1 and 1:3, between 5:1 and 1:1, between 1:1 and 1:5, between 3:1 and 1:1, between 3:1 and 1:3, including any range in between. In some embodiments, a weight ratio between the photosensitizer and the SHG particle is between 2:1 and 1:2, or about 1:1, including any range in between.

In some embodiments, the nanoparticle of the invention is referred to as stable, if at least 80%, at least 85%, at least 90%, at least 95%, at least 99%, by weight of the particles retain at least 90% of the particle size, including any range therebetween. In some embodiments, the nanoparticle of the invention is referred to as stable by weight of the particles remain between 80 and 99%, between 80 and 85%, between 85 and 90%, between 90 and 95%, between 95 and 99%, including any range in between, of the particle size within a solution. In some embodiments, the nanoparticle of the invention is referred to as stable, if at least 80%, at least 85%, at least 90%, at least 95%, at least 99%, by weight of the particles substantially retain the chemical composition thereof and/or substantially retain the capability of inducing generation of ROS.

In some embodiments, the nanoparticle of the invention is referred to as stable when between 80 and 99%, between 80 and 85%, between 85 and 90%, between 90 and 95%, between 95 and 99%, by weight of the particles remain at least 80% of the particle size within a solution (e.g., an aqueous solution) for a period of at least 1 h, at least 3 h, at least 5 h, at least 10 h, at least 24 h, at least 2 d, at least 10 d, at least 20 d, at least 1 m, at least 6 m, at least 1year, including any value and range therebetween. Each possibility represents a separate embodiment of the invention.

In some embodiments, the nanoparticle of the invention is referred to as stable, when at least 80%, at least 85%, at least 90%, at least 95%, at least 99%, by weight of the particles remain at least 90% of the particle size within a solution (e.g., an aqueous solution) for a period of at least 1 h, at least 3 h, at least 5 h, at least 10 h, at least 24 h, at least 2 d, at least 10 d, at least 20 d, at least 1 m, at least 6 m, at least 1 year, including any value and range therebetween. Each possibility represents a separate embodiment of the invention.

In some embodiments, the nanoparticle of the invention is referred to as stable, when at least 80%, at least 85%, at least 90%, at least 95%, at least 99%, by weight of the particles retain at least 80% of the initial photosensitizer content. In some embodiments, the plurality of the nanoparticles of the invention is referred to as stable by weight of the particles retain between 80 and 95%, between 80 and 85%, between 85 and 90%, between 90 and 95%, including any range in between, of the initial photosensitizer content.

In some embodiments, the nanoparticle of the invention has a core in contact with a shell, wherein the core comprises the SHG particle, and the shell comprises the spacer and the photosensitizer. In some embodiments, the shell is in a form of a continuous layer of the non-ionic water-soluble spacer molecules. In some embodiments, the non-ionic water-soluble spacer molecules are arranged within the shell in a form of a monolayer. In some embodiments, the shell is in a form of a multilayer of the non-ionic water-soluble spacer molecules. In some embodiments, the shell is in a form of a bi-layer of the non-ionic water-soluble spacer molecules.

The term “continuous layer” or the term “layer” refers to a substantially homogeneous substance of substantially uniform-thickness which maintains its physico-chemical properties (e.g., glass transition temperature, Youngs modulus, elonagation) with the entire dimensions (lengths and width dimensions) thereof. In some embodiments, each layer has a different physical structure and/or a different chemical composition. In some embodiments, each layer has the same physical structure and/or the same chemical composition. In some embodiments, the term “layer”, refers to a layer of EG, EG oligomers, or PEG, characterized by an average MW as disclosed herein.

In some embodiments, the nanoparticle of the invention is a core-shell nanoparticle. In some embodiments, a weight ratio between the shell and the core within the plurality of particles of the invention is between 1:0.1 and 1:0.001, between 1:0.1 and 1:0.01, between 1:0.1 and 1:0.05, between 1:0.05 and 1:0.01, between 1:0.01 and 1:0.001, including any range between. The term “shell” as used herein, refers to the outer portion of the particle, with a different composition than the core.

In some embodiments, the core is or comprises SHG particle, or a plurality (e.g., 2, 3, 4, 5) of SHG particles. In some embodiments, the core is or comprises a barium titanate nanoparticle, Li-niobate nanoparticle, K-niobate or any combination thereof. In some embodiments, the core comprises an outer portion facing the shell. In some embodiments, the outer portion of the core is in contact with or bound to the shell (i.e., the water soluble non-ionic spacer, as disclosed herein). In some embodiments, the outer portion is bound to the spacer (e.g., EG oligomer, or polyethylene glycol). In some embodiments, the spacer is bound to the core by a non-covalent bond and/or physical interactions. In some embodiments, the spacer is chemisorbed and/or physisorbed to the core.

In some embodiments, the nanoparticle of the invention is devoid of the photosensitizer in direct contact with the core. In some embodiments, the photosensitizer is not in direct contact with the core (e.g., Ba-titanate, and/or Li-niobate). In some embodiments, the photosensitizer is located in the shell of the nanoparticle of the invention. In some embodiments, the photosensitizer is embedded within the shell of the nanoparticle of the invention. In some embodiments, the photosensitizer is embedded within the shell of the nanoparticle of the invention and is in operable communication with the core, wherein “operable communication” is as described herein. In some embodiments, the core and the photosensitizer are spaced by the spacer. In some embodiments, the core and the photosensitizer are spaced by EG monomer, EG oligomer, or by PEG, wherein the spacer is characterized by an average MW as disclosed herein (between 50 and 1000 Da, or between 50 and 500 Da). In some embodiments, the outer portion of the core is not bound to the photosensitizer.

In some embodiments, the nanoparticle of the invention is substantially devoid of crosslinking. In some embodiments, the nanoparticle of the invention is a non-crosslinked nanoparticle.

In some embodiments, the inner portion comprises about 97%, about 98%, about 99%, about 99.9%, about 99.99%, about 99.999% of the entire weight and/or entire volume of the core. In some embodiments, the outer portion refers to the outer surface of the core. In some embodiments, the outer portion is in a form of a layer. In some embodiments, the inner portion and/or the outer portion of the core is substantially devoid of an organic compound. In some embodiments, the about 97%, about 98%, about 99%, about 99.9%, about 99.99%, about 99.999%, or between 95 and 100%, between 95 and 99%, between 97 and 100%, between 95 and 99.9%, of the entire weight of the core consists of the SHG particle.

In some embodiments, a w/w ratio between the core and the shell within the nanoparticle of the invention is between 3:1 and 1:3, between 3:1 and 1:2, between n1:3 and 1:1, between 2:1 and 1:3, between 1:1 and 1:3, including any range in between.

In some embodiments, the shell consists essentially of the spacer (e.g., PEG) and the photosensitizer. In some embodiments, the about 97%, about 98%, about 99%, about 99.9%, about 99.99%, about 99.999%, or between 95 and 100%, between 95 and 99%, between 97 and 100%, between 95 and 99.9%, of the entire weight of the shell consists of the photosensitizer and of the spacer. In some embodiments, the spacer is bound to the photosensitizer via a non-covalent bond (e.g., hydrogen bonds, van-der-Waals bond etc.) and/or physical interactions. In some embodiments, photosensitizer is embedded in the spacer.

In some embodiments, photosensitizer is stably bound to the spacer and/or to the core. In some embodiments, the term “stable” refers to the chemical stability (e.g., substantially devoid of bond cleavage) of the bond under ambient conditions (a temperature of less than 100° C., normal pressure or vacuum, and optionally ambient atmosphere), for a time period of between 1 day and 1 year including any range between.

In some embodiments, at least 80%, at least 90%, at least 93%, at least 95%, at least 97%, at least 99.9%, or 100% by weight and/or volume of the shell consist of the photosensitizer and spacer bound thereto, including any range between.

In some embodiments, at least 85%, at least 90%, at least 93%, at least 95%, at least 97%, at least 99.9%, or 100% including any range between, by weight of the nanoparticle of the invention consist of the core, the photosensitizer and the spacer bound thereto.

In some embodiments, the nanoparticle of the invention has a cubic geometry or shape. In some embodiments, the nanoparticle has an inflated or deflated shape. In some embodiments, the nanoparticle of the invention devoid of any characteristic geometry or shape. In some embodiments, the nanoparticle of the invention has a spherical shape, a quasi-spherical shape, a cubic shape, a semi-cubic shape, a quasi-elliptical sphere, a deflated shape, a concave shape, an irregular shape, or any combination thereof. One skilled in the art will appreciate that the exact shape of each of the plurality of particles may differ from one particle to another. Moreover, the exact shape of the nanoparticle of the invention may be derived from any of the geometric forms listed above, so that the shape of the particle does not perfectly fit a specific geometrical form. One skilled in the art will appreciate that the exact shape of the nanoparticle may have substantial deviations (such as at least 5%, at least 10%, at least 20% deviation) from a specific geometrical shape (e.g., a cube or a sphere).

In some embodiments, the nanoparticle of the invention is a solid nanoparticle. In some embodiments, the nanoparticle is in a form of a colloid.

In another aspect, there is provided a composition comprising a plurality of nanoparticles, wherein each of the plurality of nanoparticles comprises: (i) a SHG particle, (ii) PEG and (iii) a photosensitizer; wherein: the SHG particle and the photosensitizer are spaced by the PEG; the photosensitizer is a water insoluble photosensitizer characterized by an absorbance at a wavelength in a range between about 360 and 450 nm; and the SHG particle is barium titanate.

In some embodiments, PEG has an average molecular weight below 1000 Da, below 800 Da, below 600 Da, below 400 Da, below 200 Da, below 150 Da, and between 100 and 2000 Da, between 100 and 400 Da, between 100 and 300 Da, between 200 and 400 Da, between 200 and 600 Da, between 200 and 1000 Da, between 250 and 350 Da, between 250 and 1500 Da, between 1000 and 2000 Da, including any range in between.

In some embodiments, the PEG molecular weight is between 280 and 320 Da.

In another aspect, there is provided a pharmaceutical composition comprising the composition of the invention and a pharmaceutically acceptable carrier.

In some embodiments, the pharmaceutically acceptable carrier is also referred to as an excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Hartmann solution, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

In some embodiments, the w/w concentration of the pharmaceutical acceptable carrier within the pharmaceutical composition is between 0.1 and 99%, between 0.1 and 1%, between 1 and 10%, between 10 and 20%, between 20 and 30%, between 30 and 50%, between 50 and 60%, between 60 and 80%, or between 80 and 90%, including any range between. Each possibility represents a separate embodiment of the invention.

In some embodiments, pharmaceutically acceptable carrier comprises a buffer.

### Method for Use

According to some embodiments, there is provided a method for manufacturing the nanoparticle of the invention, wherein the method comprising contacting SHG particle, a non-ionic water-soluble spacer and a photosensitizer at appropriate conditions, thereby forming the plurality of nanoparticles of the invention. In some embodiments, the method comprises obtaining a composition comprising SHG particle, a non-ionic water-soluble spacer and a photosensitizer, and aqueous solvent.

In some the concentration of the SHG particle and the photosensitizer within the composition is independently between 100 and 1000 ug/ml, between 100 and 500 ug/ml, between 300 and 500 ug/ml, between 300 and 1000 ug/ml, between 200 and 600 ug/ml, between 300 and 700 ug/ml, between 400 and 1000 ug/ml, between 350 and 450 ug/ml, including any range in between.

In some the concentration of non-ionic water-soluble spacer within the composition is between 150 and 3000 ug/ml, between 150 and 2500 ug/ml, between 150 and 1000 ug/ml, between 300 and 1000 ug/ml, between 500 and 1500 ug/ml, between 500 and 2500 ug/ml, between 600 and 1500 ug/ml, between 800 and 3000 ug/ml, including any range in between.

In some embodiments, appropriate conditions comprise exposing the composition to a temperature suitable for obtaining the nanoparticle of the invention. In some embodiments, appropriate conditions further comprise exposing the composition to ultra-sonic waves. In some embodiments, appropriate conditions compromise exposing the composition to a temperature suitable for obtaining the nanoparticle of the invention for a period of time between 0.5 and 30 min, between 0.5 and 5 min, between 5 and 15 min, between 5 and 20 min, between 10 and 20 min, including any range in between. In some embodiments, temperature suitable for obtaining the nanoparticle of the invention is between −10 and 10° C., between −10 and 0° C., between −5 and 10° C., between −8 and −2° C., between −10 and 5° C., between −5 and 0° C. including any range in between.

In some embodiments, contacting is performed under exposing the composition to ultrasonic waves for a period of time between 0.5 and 60 min, including any range in between, and under temperature between −10 and 10° C., including any range in between.

In another aspect, there is provided a method for killing a cell of interest in a subject, comprising (i) administering an effective amount of the pharmaceutical composition of the invention to the subject, thereby internalizing the composition into a cell of interest of the subject; (ii) irradiating the cell of interest at a wavelength suitable for excitation of the SHG particle, thereby inducing cell death.

In some embodiments, step (ii) comprises irradiating a tissue of the subject comprising the cell of interest.

The term “effective amount” or “therapeutically effective amount” refers to an amount effective, at a dosages and periods of time necessary to achieve a desired therapeutic result. In some embodiments, the therapeutic result refers to reduction or elimination of at least one symptom associated with the disease or disorder, as ascribed herein. In some embodiments, the therapeutic result refers to death (e.g. by apoptosis) of at least 20%, at least 30%, at least 50%, at least 80% of the cells of interest. It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician.

In some embodiments, the method of the invention is suitable for inducing photodynamic response in the cell of interest, within a subject in need thereof. In some embodiments, the method is for performing or inducing a photodynamic therapy within the subject. In some embodiments, the method of the invention is suitable for inducing photodynamic response in the tissue of interest. In some embodiments, the method of the invention is for performing a PDT in a subject in need thereof.

In some embodiments, the method of the invention is suitable for inducing photodynamic in the tissue of interest within a subject in need thereof. In some embodiments, the method is for performing or inducing a photodynamic therapy in at least one cell (or tissue) of interest.

In some embodiments, the subject is afflicted with a pathogen-related disease (e.g. microbial infection), a cell proliferation disease (e.g. cancer), or inflammation, or any combination thereof. In some embodiments, the cell/or tissue of interest comprises a cancer cell/tissue, a pathogenic cell, or a mammalian cell/tissue infected by a pathogen, or any combination thereof. In some embodiments, the pathogen is a unicellular organism. In some embodiments, the pathogenic is selected from a bacteria, a fungi, an eucaryotic parasite (e.g. a unicellular parasite), or any combination thereof. In some embodiments, the pathogen is selected from but not limited to: a fungi, a virus, a worm, a protozoa, and a prion, including any combination thereof.

In some embodiments, pathogen-related disease is selected from but are not limited to bacterial infection, viral infection, fungal infection, parasite infection (e.g. malaria, leishmania, etc.).

In some embodiments, the subject is a mammal. In some embodiments, the mammal is a laboratory animal. Examples of laboratory animals include, but are not limited to, mice, rats, rabbits, hamsters, dogs, pigs, and monkeys. In some embodiments, the mammal is a mouse or rat. In some embodiments, the subject is a human subject.

In some embodiments, the composition of the invention is formulated for administration to the subject. In some embodiments, the method comprises administering the composition of the invention to the subject. In some embodiments, the subject is a mammal. In some embodiments, the mammal is a human. In some embodiments, the subject is in need of the composition. In some embodiments, the composition is for use in treatment of a disease or condition in humans and other mammals. In some embodiments, the subject is in need of treatment. In some embodiments, the subject is afflicted with a pathogen related disease, or a proliferation disease, or any combination thereof.

In some embodiments, the composition of the invention is formulated for administration to the cell of interest. In some embodiments, the composition is formulated for systemic administration. In some embodiments, the composition is formulated for intravenous (IV) administration. In some embodiments, the composition is formulated for inhalation administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, intravenous, subcutaneous, oral, intramuscular, intrathecal, inhaled, intracerebroventricular, intravitreal, transdermal, or intraperitoneal. In some embodiments, the composition is for use in a therapeutic method. In some embodiments, a therapeutic method is a method of treatment. In some embodiments, the composition is for use in treating a medical condition. In some embodiments, the medical condition is a condition, a disease, or a disorder.

In another aspect, there is provided a method for treating a disease or disorder within a subject, comprising (i) administering to the subject a therapeutic effective amount of the pharmaceutical composition of the invention; (ii) irradiating a tissue of interest in the subject at a wavelength suitable for excitation of the SHG particle, thereby treating the disease or the disorder within the subject. In some embodiments, therapeutic effective amount of the pharmaceutical composition of the invention is sufficient for accumulating within the cell and/or within the tissue of interest, in the subject in need thereof. In some embodiments, accumulating is in an amount sufficient for inducing photodynamic response within the cell and/or within the tissue of interest.

In some embodiments, photodynamic response encompasses killing or eliminating at least one cell of interest (such as by apoptosis) in response to a trigger (irradiation of the cell/tissue of interest by a light at wavelength suitable for excitation of the SHG particle). In some embodiments, photodynamic response encompasses excitation of the photosensitizer in response to the irradiation, wherein excitation of the photosensitizer is so as to induce generation of ROS within the cell of interest sufficient for killing or eliminating the cell of interest. In some embodiments, excitation of the photosensitizer is so as to induce generation of ROS within the cell of interest sufficient for inducing apoptosis of the cell. Apoptotic cell death can be assessed by determining the presence (or release) of pro-apoptotic factors within the cell or in the tissue of interest.

In some embodiments, irradiating the tissue of interest comprises applying radiation at a wavelength between 600 to 900 nm, between 600 and 650 nm, between 650 and 700 nm, between 700 and 750 nm, between 750 and 800 nm, between 800 and 850, between 850 and 900 nm, between 900 and 950 nm, between 950 and 1000, between 1000 and 1200 nm, between 600 and 1200 nm, between 600 and 950 nm, between 600 and 1000 nm, between 750 and 850 nm, between 750 and 770 nm, between 770 and 790 nm, between 790 and 810 nm, between 810 and 830 nm, between 830 and 850 nm, including any range in between.

In some embodiments, irradiating the tissue of interest comprises applying radiation at a wavelength disclosed hereinabove, and at a radiation dose sufficient for inducing photodynamic response within the cell/tissue of interest. In some embodiments, radiation dose is sufficient for treating or eliminating at least one symptom associated with the disease within the subject. In some embodiments, radiation dose doesn't exceed a safe dose, i.e. is below a dose associated with a toxicity within the subject.

In some embodiments, the method further comprises repeating the administration step and/or irradiation step. In some embodiments, the administration step and/or irradiation step is/are repeated for 2, 3, 4, 5,or between 2 and 10 times, including any range between.

In some embodiments, the method further comprises a preliminary step of selecting a subject suitable for treatment by the method of the invention. In some embodiments, the preliminary step is performed prior to the administration step (i.e. prior to treating the subject as disclosed herein). In some embodiments, the preliminary step comprises obtaining a sample from the subject, wherein the sample comprises a plurality of cells of interest (e.g. a blood sample, or biopsy); contacting the sample with the pharmaceutical composition or with the composition of the invention; and irradiating the sample in contact with the composition/pharmaceutical composition of the invention, wherein irradiating is performed at a wavelength as disclosed herein. In some embodiments, the preliminary step further comprises evaluating viability of the cell of interest, upon irradiating thereof, wherein reduction of cell viability (by at least 20%, at least 30%, or more) is indicative for the suitability of the subject for the treatment.

In some embodiments, the pharmaceutical composition of the invention is in a form of a solutions, a suspension, an emulsion, a tablet, a pill, a capsule, a powder, a gel, a cream, an ointment, a foam, a paste, a sustained-release formulations, and the like. In some embodiments, the pharmaceutical compositions of the invention can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc.

In some embodiments, the method comprises administering an effective amount of the pharmaceutical composition. In some embodiments, an effective amount is the human equivalent of a murine dose of between 0.1 and 10 mg/kg body weight per day. In some embodiments, the human equivalent of the murine dose depends upon the route of administration. In some embodiments, an effective amount is the human equivalent of a murine dose of between 0.1 and 10, between 0.1 and 1, between 0.1 and 0.5, between 0.5 and 1, between 0.5 and 10, between 0.5 and 2, between 1 and 5, between 5 and 10 mg/kg body weight, including any value in between.

The dosage administered will be dependent upon the age, health, and weight of the recipient, kind of concurrent treatment, if any, frequency of treatment, and the nature of the effect desired.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

As used herein, the term “about” when combined with a value refers to plus and minus 10% of the reference value (±10%). For example, a length of about 1,000 nanometers (nm) refers to a length of 1,000 nm=100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Conjugates of Nanoparticles and Photosensitizers

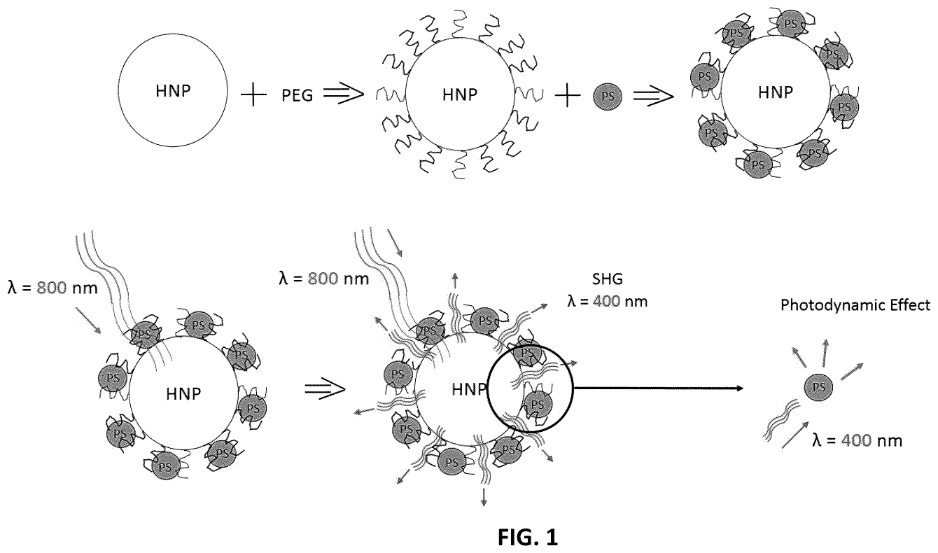

Exemplary nanoparticles of the invention were synthesized by mixing and sonicating on ice 6 mg of barium titanate (BT) (467634, Sigma-Aldrich, USA) with 6 mg of photosensitizer—either protoporphyrin IX (P8293-1G, Sigma-Aldrich, USA) or curcumin (C1386, Sigma-Aldrich, USA) in 15 ml of 6% Kollisolv® PEG-300 (PEG) (91462, Sigma-Aldrich, USA) dissolved in distilled water to derive a concentration of 400 μg/ml photodynamic therapy (PDT) particles of BT+PPIX particles or BT+Crcmn particles respectively (FIG. 1). Subsequently samples were filtered through a 0.22 μm PVDF membrane (FPV-203-013, JetBiofil, China) before.

Although BT harmonic nanoparticles are easily synthesized, they require stabilizers in hydrated environments to prevent precipitation. For effective energy transfer to occur upon light activation, the HNP and PS are required to be relatively close. By attaching PEG phospholipids to the nanoparticles, a hydrophobic environment is created surrounding the HNP for drug delivery via lipid type carriers enhancing the PS delivery. Further, the affinity of PSs for uptake by membrane bound cells and neoplastic tissues increases with an increasing degree of hydrophobicity. The PEGylated HNP's then load PS's (FIG. 1) and enhance efficiently their cellular uptake. PEGylation also improves the colloidal properties of both the HNP and PS.

### Characterization of Harmonic Nanoparticle—Photosensitizer (HNPs) Conjugates

Barium Titanate HNPs prepared by sonication and filtration for conjugation in our experiments displayed a general size of 53±9 nm and 52±9 nm for BT+PPIX and BT+Crcmn respectively, consistent with 55 nm being the ideal size of Barium Titanate HNPs for SHG efficiency.

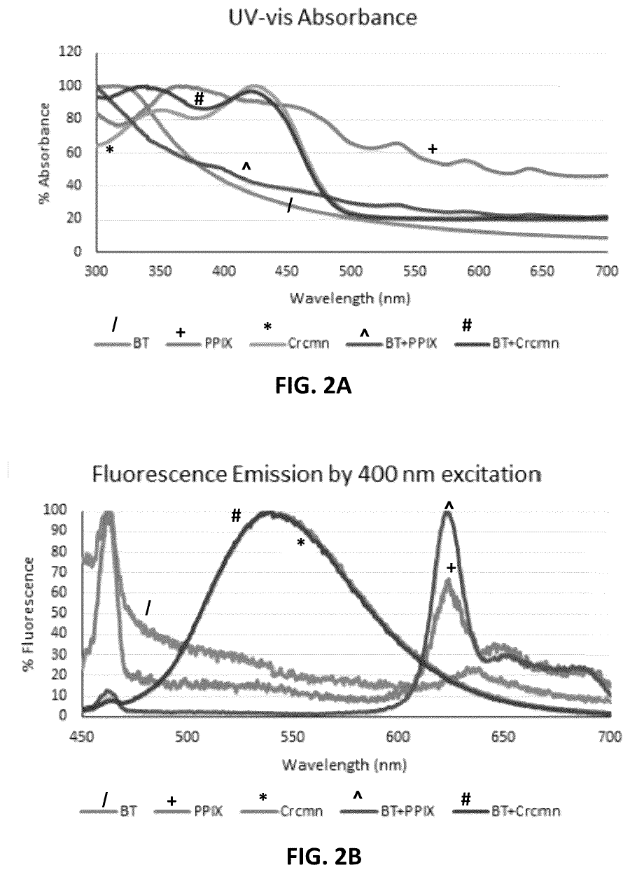

Perovskite Barium Titanate (BT) Harmonic Nanoparticles conjugated using Polyethylene Glycol (PEG) to either FDA approved Protoporphyrin IX (PPIX) or Curcumin as PSs were analyzed for their spectral properties (FIG. 2A) PPIX by itself showed an absorbance saturation over 369-400 nm with a broad peak at 365 nm. Curcumin by itself showed an absorbance saturation over 415-430 nm with a peak at 424 nm. BT by itself did not show any absorbance in the 350-400 nm range. After conjugation with PPIX the absorbance profile of BT demonstrated a small peak around 400 nm corresponding to that of the photosensitizer PPIX by itself indicating a proximity of particles. Conjugation with Curcumin shifted the absorbance profile of BT demonstrating an additional peak at 340 nm besides the already reported 424 nm peak for Curcumin alone.

BT by itself upon excitation with 400 nm showed a fluorescence emission maximum at 463 nm (FIG. 2B). PPIX by itself after excitation with 400 nm showed 2 fluorescence emission maxima at 462 nm and 624 nm (FIG. 2B). After conjugation with BT the fluorescence emission profile of PPIX demonstrated a small peak around 463 nm corresponding to that of the photosensitizer PPIX by itself and an enhanced peak at 624 nm indicating a proximity of nanoparticles with the photosensitizer. Curcumin by itself upon excitation with 400 nm showed a fluorescence emission maximum at 540 nm (FIG. 2B). Conjugation with BT did not affect the fluorescence emission profile of Curcumin demonstrating a similar peak at 540 nm as was the case for the photosensitizer alone.

### Example 2

### Electro-Optics

Spectrometry—Spectral Analysis was done using V-730 UV-Visible Spectrophotometer (Jasco, USA) and analyzed using Spectra Manager™ Suite Spectroscopy Software (Jasco, USA).

Laser Setup—Laser was generated using Millennia® Pro s Series 5SJSPG (Spectra physics, USA) laser setup to generate a 546 nm continuous wave laser which was passed through an ultra-fast oscillator Femtosource® Scientific 20 (Femtolasers, Inc.) to generate the Near Infrared (800 nm) pulsed wave (pulse frequency 76.9 MHz) beamline. The beamline was appropriately arranged to provide real-time power readings using a beam splitter. Power measurements were recorded using a compact power meter PM100D (Thorlabs, Inc.) with S370C thermal power head (Thorlabs, Inc.)).

### Cell Culture

S. aureus (ATCC 25923) were grown on Brain Heart agar plates (BHA, Acumedia, Lansing, MI, USA) for 24 hours. Grown cells were transferred into Brain Heart broth (BH, Acumedia, Lansing, MI, USA) and incubated at 37±1° C. with shaking at 170 rpm until reaching the absorbance A=0.10±0.02 at 660 nm, corresponding to a final concentration of 108 cells/ml and diluted appropriately to the final concentration of 10³-10⁴ cells/ml with commercially available sterile 0.9% saline solution before the light and dark phototoxicity assays.

### Light Phototoxicity

200 μl bacterial suspensions in 0.9% sterile saline solution (10³-10⁴ cells/μl) were incubated with 20 μl of PDT nanoparticle conjugates (400 μg/ml) under laser (800 nm, 232-228 mW,76.9 MHz) with shaking for 10 minutes. Subsequently, aliquots of each sample (100 μl) were spread over BHA plates with a Drigalski spreader and incubated at 37° C. for 24 h. The CFUwere counted using a colony counter Scan 500.

### Dark Toxicity

200 μl Cell suspensions in 0.9% saline (10³-10⁴ cells/ml) in sterile saline solution were incubated with 20 μl of PDT nanoparticle conjugates (400 μg/ml) in the dark with shaking for 30 minutes. Subsequently, aliquots of each sample (100 μl) were spread over BHA plates with a Drigalsky spreader and incubated at 37° C. for 24 h. The colony forming units (CFU) were counted using a colony counter Scan 500 (Interscience, Saint-Nom-la-Breteche, France).

Second Harmonic Generation of Harmonic Nanoparticles and Activated Emission from Conjugated Photosensitizers Using Pulsed NIR Irradiation

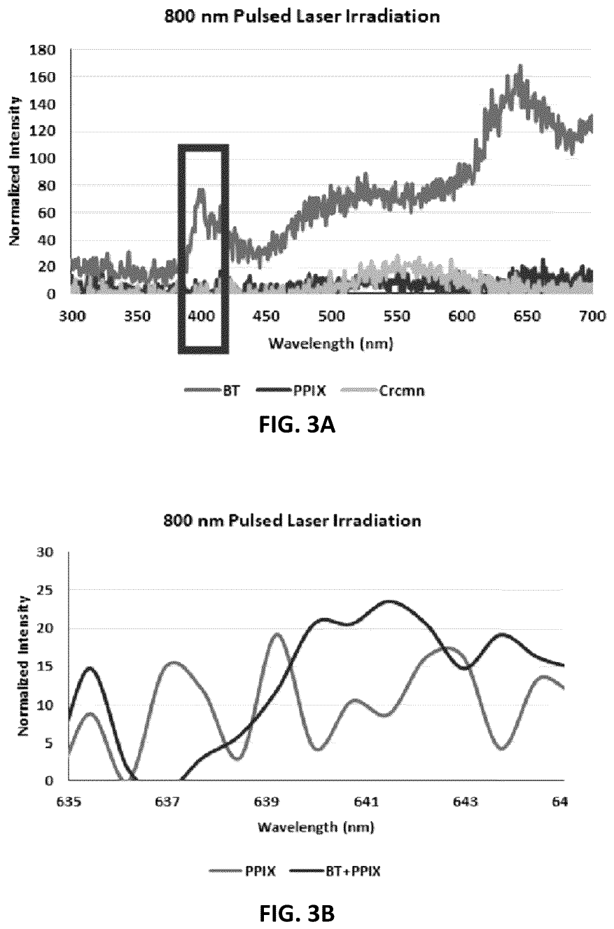

A femtosecond pulsed laser irradiation at 800 nm on BT by itself showed an emission peak at400 nm demonstrating the Second Harmonic Generation from harmonic nanoparticles (FIG. 3A) while the photosensitizers on their own without conjugation did not show any such response. Conjugation of PPIX with harmonic nanoparticles of BT activated a pronounced emission peak at 641.5 nm which was absent without conjugation (FIG. 3B). Similarly, conjugation of Crcmn with harmonic nanoparticles of BT activated an emission peak at 559.8 nm which was absent without conjugation (FIG. 3B).

NIR Photodynamic Effect of HNP-PS Conjugates on S. aureus

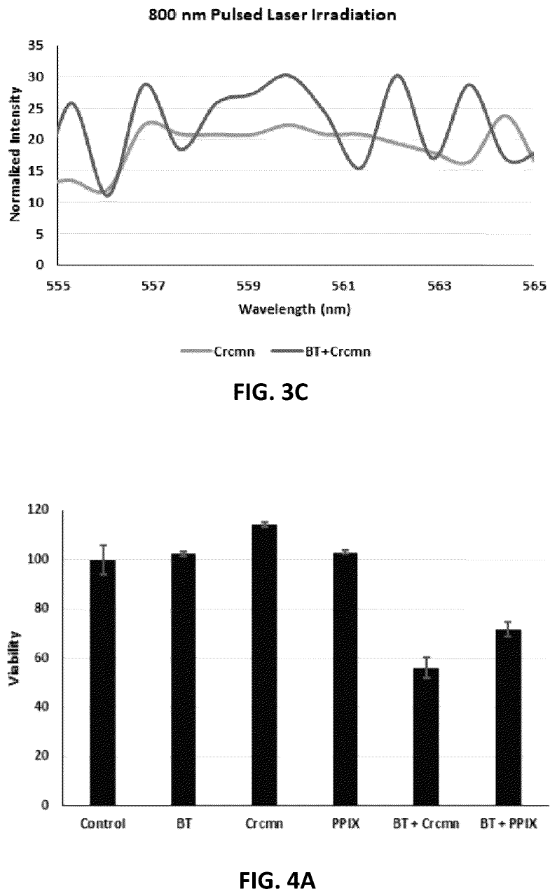

Dark toxicity tests of the prepared HNP-PS conjugates demonstrated that viability of pathogenic S. aureus after incubation with either BT+PPIX or BT+Crcmn with 30 minutes of dark incubation was reduced by 28.4±3.0% and 44.1±4.2% respectively (FIG. 4A) as compared to those of the untreated control. Samples subjected to dark conditions with either BT alone or the PPIX alone didn't affect the viability significantly as compared to control. However, Cromn alone showed 14.2±1.1% increase in viability compared to the control.

The photodynamic effect, using 20 μl (concentration 400 μg/ml) of either BT+PPIX or BT +Crcmn with 10 minutes of exposure to pulsed NIR (800 nm) laser radiation reduced the viability of 200 μl (concentration 10³-10⁴ cells/ml) of treated S. aureus samples by 77.3±9.7% and 38.4±6.1% respectively as compared to those of the untreated control (FIG. 4B).

Laser exposure alone reduced the viability of treated samples by 28.8±0.1%. BT with laser exposure increased the viability of treated samples by 24.7±0.4%. PPIX alone with 10 minutes laser exposure didn't have a significant effect on S. aureus viability, reducing it by 20.4±2.5% similar to that of the laser irradiation alone. Curcumin alone with 10 minutes laser exposure increased the viability of treated samples by 20.2±1.4%. This indicates that the proximity of conjugation to the HNP activates the photosensitizers to generate the desired antibacterial photodynamic effect in the NIR spectral range.

The average power of laser exposure was measured during the course of the experiments. The readings were normalized to provide a similarity of energy delivery among the samples of nanoparticles and conjugates for the antibacterial photodynamic effect under test.

These studies indicate that simply coupling SHG capable nanoparticles, stabilized in suspension with conventional PSs, can directly improve the treatment efficacies of PDT without the requirement of new chemical hybrids or syntheses.

In addition, by incorporating appropriate SHG capable BT harmonic nanoparticles into the PDT process, commonly used photosensitizers conventionally responsive only to visible light spectrum (illustratively Protoporphyrin IX and Curcumin respectively) can be made photodynamically reactive to deeper penetrating Near-Infrared light.

Moreover, the experiments demonstrate that Protoporphyrin IX significantly performs more efficiently than Curcumin for use in SHG mediated Near Infrared Photodynamic therapy of visible light reactive photosensitizers.

### Example 3

### The Effect of the (PEG) Ligand Length on the SHG and PDT Effect

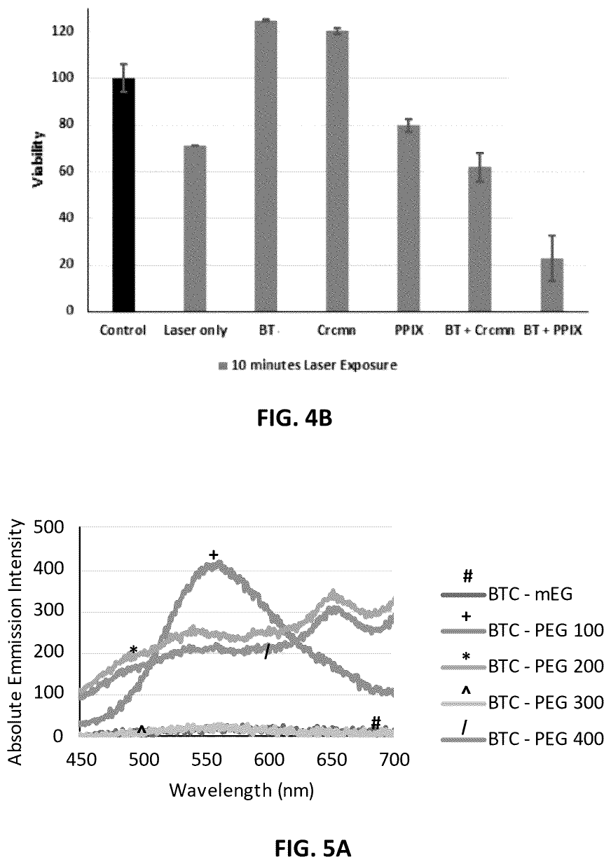

The conjugation between the BT and the photosensitizer (either Curcumin or Protoporphyrin IX, BT+Crcmn or BT+PPIX respectively) was performed in the presence of five different ligand length, based on the synthesis procedure mentioned above. Five ligands were tested, ethylene glycol monomer PEG100, PEG200, PEG300 and PEG400, and their SHG ability was tested. Spectral emission upon pulsed 800 nm laser excitation was examined, for the five different BT+Crcmn and BT+PPIX conjugates (FIG. 5A and FIG. 5B). In both cases conjugates comprising PEG 100 demonstrated the best spectral performance.

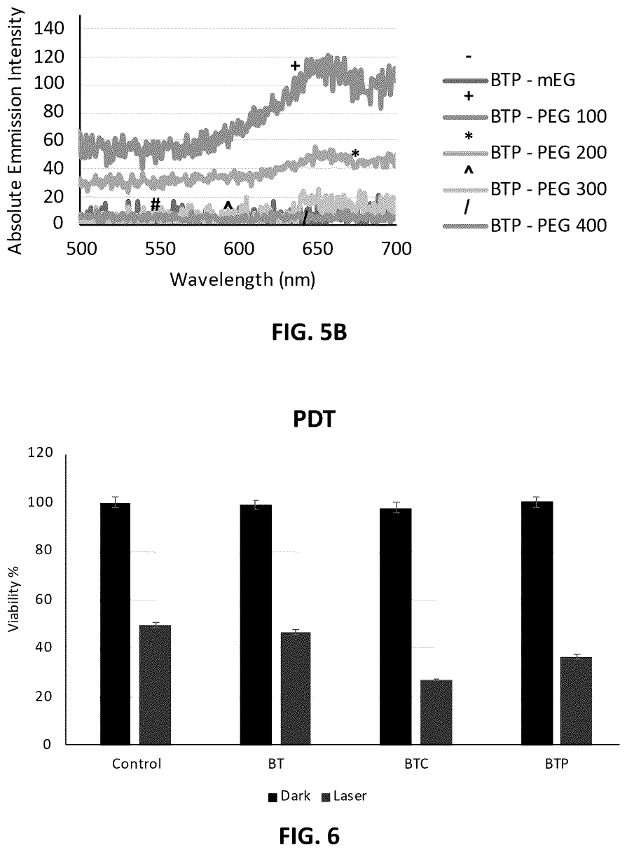

MCF7 cells were then incubated for 1 h with the either BT+Crcmn and BT+PPIX comprising PEG100, in both cases a reduction in viability was seen 73% and 64% respectively (FIG. 6). Accordingly, it is presumed that the nanoparticles of the invention can be successfully utilized for performing or inducing PDT within a subject in need thereof (such as for treating proliferative disease or infectious diseases).

Furthermore, the inventors successfully synthesized Li-niobate based nanoparticles of the invention (using PEG as the water-soluble spacer and protoporphyrin as the photosensitizer), as disclosed hereinabove for the Ba-titanate based particles. Li-niobate based nanoparticles exhibited similar spectral properties as Ba-titanate (Li-niobate excitation induced expiation of the photosensitizer, which will presumably result in ROS generation). Accordingly, it is postulated that perovskite-based nanoparticles of the invention are suitable for performing or inducing PDT within a subject in need thereof (such as for treating proliferative disease or infectious diseases).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising a plurality of nanoparticles, wherein each of said plurality of nanoparticles comprises (i) a second harmonic generation (SHG) particle, (ii) a non-ionic water-soluble spacer, and (iii) a photosensitizer; wherein:
the SHG particle is configured to emit a plurality of photons at a wavelength range of between 360 and 450 nm;
said photosensitizer is a water insoluble photosensitizer characterized by an absorbance range at least partially overlapping with said wavelength range;
and wherein said photosensitizer is in operable communication with the SHG particle to allow excitation of said photosensitizer by said plurality of photons.

2. The composition of claim 1, wherein said operable communication comprises a distance between the SHG particle and the photosensitizer suitable for excitation of said photosensitizer by said plurality of photons.

3. The composition of claim 2, wherein said SHG particle and the photosensitizer are spaced by the non-ionic water-soluble spacer; and wherein said distance is predetermined by the molecular weight of said water-soluble spacer.

4. The composition of claim 3, wherein said molecular weight is between 50 and 1000 Da.

5. The composition any claim 1, wherein said water-soluble spacer comprises ethylene glycol (EG), EG oligomer, or polyethyleneglycol (PEG).

6. The composition of claim 1, wherein said SHG nanoparticle is selected from, barium titanate, lithium niobate and potassium niobate, or any combination thereof.

7. The composition of claim 1, wherein said photosensitizer is selected from curcuminoid-based photosensitizer, porphyrin-based photosensitizer or phthalocyanines-based photosensitizer, and any combination thereof.

8. The composition of claim 7, wherein said porphyrin-based photosensitizer is protoporphyrin IX including any salt thereof.

9. A composition comprising a plurality of nanoparticles, wherein each of said plurality of nanoparticles has a core in contact with a shell; wherein:
said core comprise a SHG particle configured to emit a plurality of photons at a wavelength range of between 360 and 450 nm;
said shell comprises a water-soluble non-ionic spacer and a photosensitizer; and
said photosensitizer is a water insoluble photosensitizer characterized by an absorbance range at least partially overlapping with said wavelength range;
said photosensitizer is spaced from said core by water-soluble non-ionic spacer to allow excitation of said photosensitizer by said plurality of photons.

10. The composition of claim 9, wherein said photosensitizer is selected from curcuminoid-based photosensitizer or porphyrin-based photosensitizer, and any combination thereof.

11. The composition of claim 10, wherein said porphyrin-based photosensitizer is protoporphyrin IX including any salt thereof.

12. The composition of claim 9, wherein said water-soluble non-ionic spacer has an average molecular weight between 50 and 1000 Da.

13. The composition of claim 9, wherein said water-soluble non-ionic spacer comprises ethylene glycol (EG), EG oligomer, or polyethyleneglycol (PEG).

14. The composition of claim 1, wherein a weight ratio between said photosensitizer and said non-ionic water-soluble spacer within said composition is between 1:150 and 1:3500.

15. The composition of claim 1, wherein a weight ratio between said photosensitizer and said SHG particle within said composition is between 5:1 and 1:5.

16. The composition of claim 1, wherein each of said plurality of nanoparticles is suitable for inducing a photodynamic response in a cell.

17. (canceled)

18. (canceled)

19. The composition of claim 1 further comprising a pharmaceutically acceptable carrier.

20. A method for killing a cell of interest in a subject, comprising (i) administering an effective amount of the pharmaceutical composition of claim 19 to said subject, thereby internalizing said nanoparticle into the cell of interest; (ii) irradiating a tissue of said subject comprising said cell of interest at a wavelength suitable for excitation of said barium titanate, thereby inducing death of said cell of interest.

21. (canceled)

22. The method of claim 20, wherein said subject is afflicted with a pathogen-related disease, a cell proliferation related disease, or any combination thereof.

23. The method of claim 20, wherein said cell of interest comprises a cancer cell, a pathogenic cell, a cell infected by a pathogen, an immune cell or an activator thereof, or any combination thereof.

24. (canceled)
